(19) United States
(12) Patent Application Publication
(10) Pub. No.: US 2005/0153321 A1
(43) Pub. Date: Jul. 14, 2005

(54) Libraries of multiple-ligand-conjugated nucleic acids

(76) Inventor: James Anthony Saba, Toledo, OH (US)

Correspondence Address: JAMES SABA, 2934 111TH ST., TOLEDO, OH, 43611, US

(21) Appl. No.: 10/986,951

(22) Filed: Nov. 15, 2004

Related U.S. Application Data
(60) Provisional application No. 60/532,999, filed on Dec. 30, 2003.

(51) Int. Cl. C12Q 1/68; C07H 21/04

(52) U.S. Cl. 435/6; 536/24.3

(57) ABSTRACT

Disclosed herein are combinatorial libraries of multiple-ligand-conjugated nucleic acids. Each library member is affixed to a support such as a microarray chip or encoded bead. The nucleic acid of each member is an oligonudeotide hairpin or multiple hybridized oligonucleotides, and preferably comprises a small bioactive nucleic acid. Small bioactive nucleic acids include decoys, antisense nucleic acids, and immunostimulators.

### CROSS-REFERENCE TO RELATED APPLICATION

This application claims the benefit of U.S. Provisional Application Ser. No. 60/532,999 filed Dec. 30, 2003.

### BACKGROUND OF INVENTION

Small bioactive nucleic acids (decoys, antisense nucleic acids, and immunostimulators) have vast technical, cosmetic, and therapeutic potential in that they can modulate core cellular metabolism and precisely effect DNA sequence alteration and recombination. The present invention teaches novel combinatorial libraries of multiple-ligand-conjugated nucleic acids which are preferably arrayed and primarily useful in discovering combinations of ligands which facilitate the functioning of small bioactive nucleic acids.

Neri, et al (US/20040014090, Jan. 22, 2004) teach a soluble combinational library wherein each member consists of multiple hybridized ligand-conjugated oligonucleotides, and its utility in discovering bioactive ligand combinations. They teach the oligonucleotides as inert scaffolds for combining ligands, not as bioactive agents. Considering the vast potential of small bioactive nucleic acids, it's unimaginable they had conceived of, or thought obvious incorporating these and not have taught so. They also teach identifying isolated target-binding library members by addressing them to arrayed oligonucleotides, thus producing a noncovalently affixed array of multiple-ligand-conjugated nucleic acids. They do not teach a library whose every member is covalently affixed on or within a support, particularly where the support is a bead. Furthermore, they did not teach the advantageous durability and resilience of multiple-ligand-conjugated oligonudeotide hairpins.

Vargeese, et al (US/20040110296, Jun. 10, 2004) teach solitary bioactive multiple-ligand-conjugated nucleic acids (their FIGS. 41 & 42). They specifically teach oligonucleotide hairpins in which only one of the two terminal nucleotides is conjugated to a ligand. Most importantly, it is clear they as Neri et al have not appreciated the enormous utility of a support-affixed library, particularly where the hybridization has effected ligand combinations. Indeed their multiple-ligand-conjugated nucleic acids appear ill equipped to be covalently affixed to a support, particularly so that the ligands are grouped and positioned distal the support.

### BRIEF SUMMARY OF INVENTION

Disclosed herein are combinatorial libraries of multiple-ligand-conjugated nucleic acids. Each library member is affixed to a support such as a microarray chip or encoded bead. The nucleic acid of each member is an oligonucleotide hairpin or multiple hybridized oligonucleotides, and preferably comprises a small bioactive nucleic acid. Small bioaclive nucleic acids include decoys, antisense nucleic acids, and immunostimulators.

### BRIEF DESCRIPTION OF DRAWINGS

FIG. 1. An oligonucleotide hairpin decoy whose terminal nucleotides are conjugated to ligands which are facilitating the decoy's binding, perhaps to a transcription factor.

FIGS. 2A & 2B. Examples of a multiple-ligand-conjugated oligonucleotide hairpin whose ligands have bound to one or multiple target proteins.

FIG. 3. Synthesis of an array of multiple-ligand-conjugated oligonucleotide hairpins.

### DETAILED DESCRIPTION OF INVENTION

A “polynucleotide” consists of polymerized nucleotides, and an “oligonucleotide” is a polynucleotide of less than 250 nucleotides. A “nucleic acid” consists a solitary oligonucleotide, or multiple hybridized oligonucleotides. In addition to the naturally occurring nucleotides there are numerous known and conceivable denvatizations and polymerized combinations thereof. Noteworthy are peptide nucleic acids (PNA).

Certain oligonucleotides can intrahybridize to form hairpins, each of whose duplex stem portion comprises at least four contiguous base-pairs. The termini of such a hairpin can deviate a few nucleotides from being exactly flush, and the duplex stem portion may comprise a few mismatched and/or unmatched nucleotides. Furthermore, the loop nucleotides of a hairpin may be replaced by a chemical linker (5) which would be considered part of a highly derivatized internal nucleotide.

A “polypeptide” consists of polymerized amino acids. Numerous unnatural derivations with modifications to both the amino acids and their linkages are known or conceivable. Amino acids lack complementarity and consequently are unable to match-up (base-pair) as can most nucleotides.

A “protein” comprises one or more polypeptides, and may also comprise one or more polynucleotides, lipids, and/or carbohydrates (also termed saccharides and polysaccharides). Thus ribonucleoproteins and glycoproteins are proteins. For the purposes herein the largest protein is a ribosome.

A “target” is a protein. Examples thereof are cell membrane-associated proteins particularly receptors; nucleic add processing proteins; intracellular transport proteins; and viral and cellular structural proteins.

A “ligand” is a group of covalently bound atoms which alone or in association with one or more other ligands binds to, or is anticipated to bind to a target. Usually a ligand can be distinguished from its conjugated nucleotide due to being coupled via a commonly used bond such as an amide, ester, or disulfide. In such cases the ligand (including any linking group) is at least 200 Daltons MW. When distinguishing ligand is not so clear the terminal nucleotide-ligand combination is at least 850 Daltons MW. A ligand does not comprise a nucleotide. A target-bound ligand usually contacts a target at one location, yet certain ligands can contact a target at multiple sites, or even contact multiple targets. Binding is usually noncovalent, yet covalent modification of the target is conceivable.

Conjugation of ligand to a terminal nucleotide of an oligonucleotide is well known. One method of forming such a conjugate is to covalently couple a preformed ligand and oligonucleotide. Synthesizing large numbers of different ligand-conjugated oligonudeotides is readily accomplished by step-wise synthesis of the ligand and/or oligonucleotide, such as by portioning and mixing (also termed split-pool or split-and-mix), microfluidics, or photolithography (6-8). Conjugation may be via a linking group, perhaps functioning as a spacer and/or as a point of in vitro or in vivo cleavage. Such cleavage may be prodrug activating.

Small bioactive nucleic acids include decoys, antisense nucleic acids, and immunostimulators (9). A nucleic acid decoy binds to a site on a protein which normally binds a native nucleic acid, usually inhibiting the protein's function (10-14). Important types of proteins bound by nucleic acid decoys include those involved in DNA replication, recombination, repair, and transcription; RNA processing and translation; and viral nucleic acid packing. An antisense nucleic acid functions via sequence-specific hybridization to a viral or cellular nucleic acid such that it sterically hinders the functioning of and/or effects the processing of this viral or cellular nucleic acid (15-21). Important variations of antisense nucleic acids include RNase L activating 2-5A chimerics; RNA-DNA chimeric mutational vectors; triplex-forming oligonucleotides; ribozymes; and RNA interferers (RNAi) such as small interfering RNAs (siRNA) and small hairpin RNAs (shRNA).

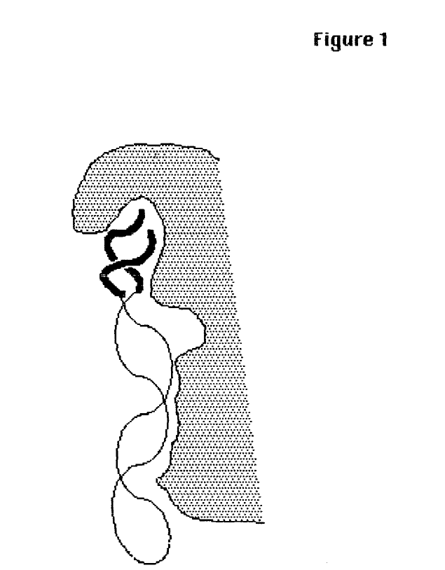

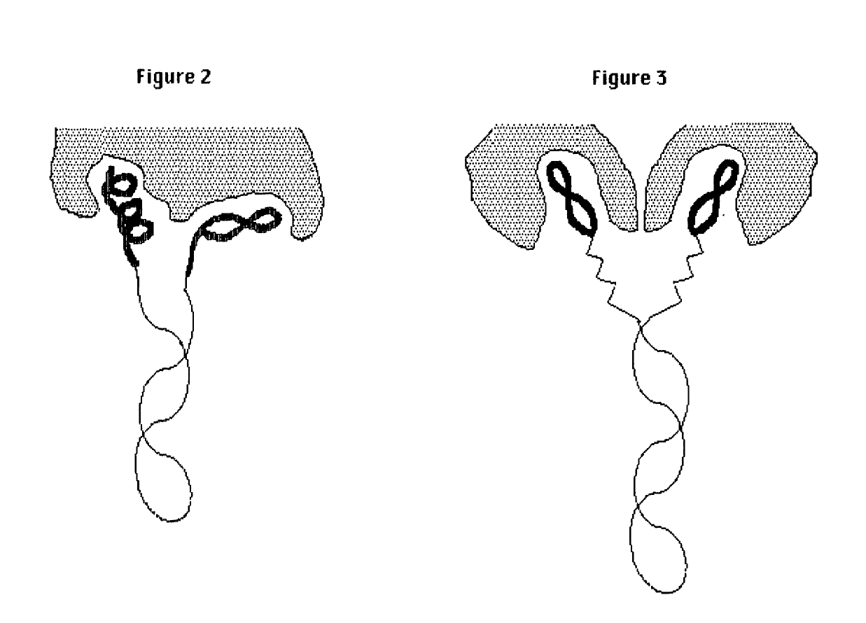

FIGS. 1, 2 & 3 exemplify a target-bound oligonucleotide hairpin whose terminal nucleotides are conjugated to separate ligands. In these sketches the smoothly curved narrow lines represent oligonudeotides, while the broad lines represent polymeric ligands such as polypeptides or polysaccharides. The stippled forms represent bound target proteins.

FIG. 1 depicts a nucleic acid decoy whose set of conjugated ligands is facilitating the decoy's binding, perhaps to a cellular transcription factor, viral transactivator, or bacterial amnioacyl-tRNA synthetase. Other important ways a ligand set can facilitate the functioning of a small bioactive nucleic acid include enhancing viral or cellular specificity and penetration, directing intracellular localization, and modulating a cellular signaling pathway.

In FIG. 2 the conjugated ligands have bound to one and the same target protein in a cooperative fashion. Perhaps one ligand was previously known to bind the target and thereby induce a cellular response, and the other was selected for its capacity to increase the specificity and/or binding tenacity of this known target binder. Alternatively, perhaps the ligands are binding to a site on a protein which interfaces with another protein, and are thus capable of sterically hindering protein-protein association (22).

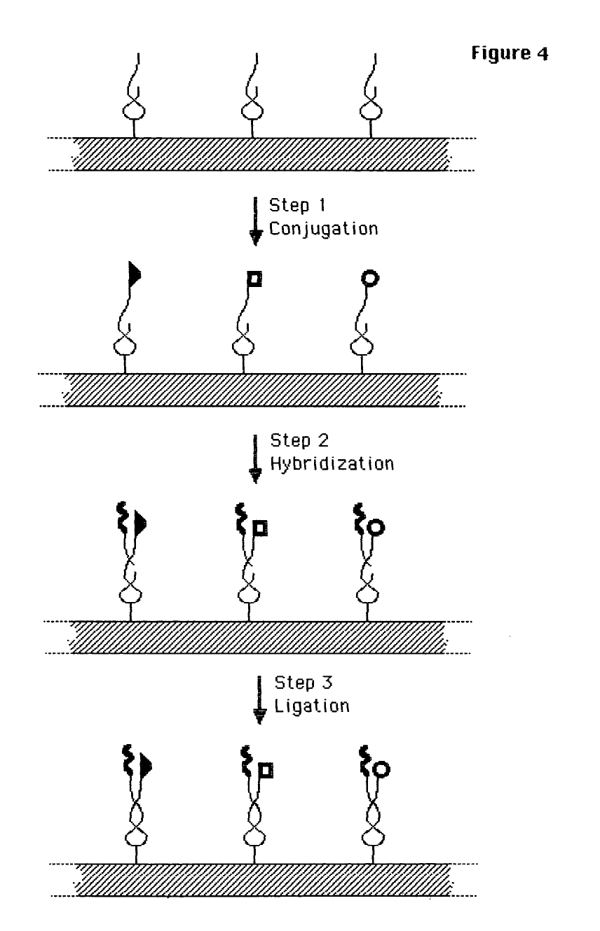

In FIG. 3 two identical ligands, conjugated via linkers, have bound to two identical targets so as to bring them into association, perhaps inducing a cellular response.

Antigenic ligands are recognized and bound by immunological proteins, particularly T cell receptors and antibodies. A small bioactive nucleic acid conjugated to antigenic ligands can alter immune system functioning (23). For example, enhancing immune cell function during immunization, or suppressing or killing immune cells to induce tolerance for transplantation or treatment of autoimmune disease. A ligand or ligand set may function as a continuous or discontinuous epitope.

Of essence is the creation and screening of a library of multiple-ligand-conjugated nucleic acids. Such a library contains at least ten and preferably more than a hundred different members. For clarity in describing a library only one of each different member is being referred to, yet in practice each different member occurs in multiple.

Each member of a library is affixed on or within a support. Frequently library members are arrayed utilizing a glass slide or silicon wafer (24-26), and the surfaces of these supports are often derivatized chemically and/or thinly coated or spotted with a three-dimensional gel. Interesting examples the novel types of random and nonrandom arrays are continually being disclosed, examples thereof utilize solid or hollow fibers (28).

Particles, often encoded and randomly arrayed, are also useful supports. While beads are commonly used as particles, exceptionally small crystalline particles such as carbon nanotubes have utility. Preferably each library member is affixed to an encoded particle (29-31).

FIG. 4 exemplifies fabricating an arrayed library of multiple-ligand-conjugated oligonucleotide hairpins. For clarity, only a section of the array is depicted. We start with three identical partial oligonucleotide hairpins covalently affixed to three array loci. Fabricating such an array has been described (24-26). Also previously described is the use of photodeavable or chemically cleavable linkers to affix an oligonucleotide to a support (32,33) and if desired these can be incorporated. In step 1, one terminus of each oligonucleotide is conjugated to a different ligand. In Step 2, identical ligand-conjugated oligonucleotides are hybridized to those arrayed to effect ligand combinations. Finally, in Step 3 the resultant hybrids are covalently fused via ligation to form an exceptionally durable and resilient array of multiple-ligand-conjugated oligonucleotide hairpins which have identical nucleic acids but different sets of ligands. Of course hybridization is not required in forming such an array, for example ligands could be conjugated to preformed hairpins.

Note the significant potential for diversity in that the common ligand utilized in Step 2 can be changed. For example, assume we have fabricated 1000 identical biochips, each arrayed with 100,000 identical partial oligonucleotide hairpins conjugated to different ligands. Simply by utilizing a different secondary ligand for each biochip, a hundred million (100,000,000) different ligand combinations are synthesized.

A library whose members have identical nucleic acids but different sets of ligands as that in FIG. 4 is valuable in discovering or investigating combinations of ligands which facilitate the functioning of a small bioactive nucleic acid, or combinations of ligands which themselves have certain structural or functional characteristics. Alternatively the members of a library may have identical ligand sets but different nucleic acids; or different ligand sets and different nucleic adds.

A library may also have diagnostic utility. For example one could simultaneously assay various cancer cell transcription factors utilizing an array of multiple-ligand-conjugated hairpin decoys, each of which was previously known to specifically bind a particular transcription factor. Another example of diagnostic utility would be to assay a patient's antibodies via an array of multiple-ligand-conjugated hairpins comprising known discontinuous epitopes.

It is to be appreciated that any library of multiple-ligand-conjugated nucleic acids wherein each member is affixed on or within a support and comprises a small bioactive nucleic acid, is novel and embodied in the present invention.

It is also to be appreciated that any library of multiple-ligand-conjugated nucleic acids wherein each member is covalently affixed on or within a support is novel an embodied in the present invention. Examples thereof are the arrays in FIG. 4 prior to and after ligation.

Various processes for screening libraries are known or conceivable. One relatively simple array-based screening process involves contacting an array with a target, and then determining the loci to which the target bound (26).

Another array-based screening process involves noncovalently arraying library members on a surface, and then covering this array with a monolayer of cells (27). A variation of this would be to covalently array the library members, optionally via a cleavable linker.

Noteworthy is a process in which two arrays are brought into contact, and optionally involves liberation of support-affixed oligonucleotides (33).

An example of a screening process utilizing particles involves covering a confluent layer of cells with a thin layer of agarose within which beads are thinly dispersed. Photocleavage the library members from the beads releases them to local cells, and subsequent identification of cells whose function is altered reveals the active bead (31). Interesting, in this example each library member is covalently affixed to a support, the bead, and also noncovalently arrayed within the three-dimensional gel support.

An important screening technique involves modulating cellular expression of a detectable protein, such as green fluorescent protein or luciferase (27,34,35). Combining this technique with whole animal fluorescence (36) one could examine targeting to a particular cell, tissue or organ.

Considerable effort has been given to make this discloser clear and concise. Thus modifications and variations are certain and it is intended that these be included within the scope of present invention as defined by the claims.

### References

The following artides, and often the references they contain, more fully describe the state of the art and teach material and methods applicable to the present invention. Thus to avoid needless repetition of these materials and methods each of these articles is incorporated by reference.
1) Encoded self-assembling chemical libraries (ESACHEL). Neri, et al US/20040014090 Jan. 22, 2004
2) Conjugates and compositions for cellular delivery. Vargeese, et al US/20040110296 Jun. 10, 2004
3) Ligand-conjugated polynucleotides and microarrays of combinatorial libraries thereof. (see references therein) Saba, J The General Science Journal (Walter Babin Editor) Jan. 17, 2004 hftp://www.wbabin.net/saba/saba12.htm
4) Ligand-conjugated polynucleotides and microarrays of combinatorial libraries thereof. (update) Saba, J The General Science Journal (Walter Babin Editor) Jun. 22, 2004 http://www.wbabin.net/saba/saba14.htm
5) Incorporation of a non-nucleotide bridge into hairpin oligonudeotides capable of high-affinity binding to the Rev protein of HIV-1. Nelson, et al Biochemistry. Apr. 23, 1996;35(16):5339
6) Stepwise solid-phase synthesis of peptide-oligonucleotide conjugates and supports therefor. Antopolskii, et al US/20040054161 Mar. 18, 2004
7) Parallel synthesis of PNA-peptide conjugate libraries. Awasthi S K, et al Comb Chem High Throughput Screen. May 2002;5(3):253-9
8) Large scale photolithographic solid phase synthesis of polypeptides and receptor binding screening thereof. Pirrung, et al U.S. Pat. No. 5,143,854
9) Oligonudeotides: Antisense, RNAi, triple-helix, gene repair, enhancer decoys, CpG, and DNA Chips. Annals of the New York Academy of Sciences, Vol 1002, 2004 Editors Stein C A, Cho-Chung Y S & Gewirtz, A M New York Academy of Sciences
10) New trends in the development of transcription factor decoy (TFD) pharmacotherapy. (review) Gambari, R Curr Drug Targets. July 2004;5(5):419-30
11) Subcellular localization as a limiting factor for utilization of decoy oligonucleotides. Bene, et al Nucleic Acids Res. Oct. 21, 2004;32(19):e142
12) Cellular delivery of a double-stranded oligonucleotide NFkappaB decoy by hybridization to complementary PNA linked to a cell-penetrating peptide. Fisher, et al Gene Ther. August 2004;11 2004(16):1264-72
13) Specific covalent binding of a NF-kappaB decoy hairpin oligonucleotide targeted to the p50 subunit and induction of apoptosis. Lesage, et al FEBS Lett. Jul. 17, 2003;547(1-3):115-8
14) Therapeutic effect of in vivo transfection of transcription factor decoy to NF-kappaB on septic lung in mice. Matsuda, et al Am J Physiol Lung Cell Mol Physiol. Aug. 6, 2004
15) Antisense hairpin loop oligonucleotides as inhibitors of _expression of multidrug resistance-associated protein 1: their stability in fetal calf serum and human plasma. Rebowski, et al Acta Biochim Pol. 2001;48(4):1061-76
16) Single-stranded oligodeoxynucleotide mutational vectors. Metz, et al US/20040023392 Feb. 5, 2004
17) Site-directed recombination via bifunctional PNA-DNA conjugates. Rogers, et al Proc Natl Acad Sci USA. Dec. 24, 2002;99(26):16695-700
18) Selective RNA deavage by isolated RNase L activated with 2-5A antisense chimeric oligonucleotides. (review) Silverman, et al Methods Enzymol. 2000;313:522-33
19) Small interfering RNAs and their chemical synthesis. Micura R. Angew Chem Int Ed Engl. Jul. 2, 2002;41(13):2265-9
20) Enzymatic nucleic acid peptide conjugates. Beigelman, et al US/20030148928 Aug. 7, 2003
21) Triplex-forming oligonucleotides: principles and applications. (review) Vasquez K M, et al Q Rev Biophys. February 2002;35(1):89-107
22) Mapping of Synergistic Components of Weakly Interacting Protein-Protein Motifs Using Arrays of Paired Peptides. Espanel, et al J. Biol. Chem. April 2003;278(17)15162-15167
23) Immunostimulatory polynucleotidelimmunomodulatory molecule conjugates. Carson, et al US/20040006010 Jan. 8, 2004
24) DNA microarrays with stem-loop DNA probes: preparation and applications. Broude, et al Nucleic Acids Res. 29, e92 2001
25) Investigation of the multiple anchors approach in oligonucleotide microarray preparation using linear and stem-loop structured probes. Bordoni, et al Nucleic Acids Res. Apr. 15, 2002;30(8):E34-4
26) DNA microarrays with unimolecular hairpin double-stranded DNA probes: fabrication and exploration of sequence-specific DNA/protein interactions. Wang, et al J Biochem Biophys Methods. Mar. 28, 2003;55(3):215-32
27) RNAi microarray analysis in cultured mammalian cells. Mousses, et al Genome Res. October 2003;13(10):2341-7
28) Selectively hybridizable substance immobilization fiber, fiber array comprising bundle of such fibers, selective hybridizing method, device therefor, and base. US/20040096169 Sone, et al May 20, 2004
29) Design, synthesis, screening, and decoding of encoded one-bead one-compound peptidomimetic and small molecule combinatorial libraries. (review) Liu, et al Methods Enzymol. 2003;369:271-87
30) Target analyte sensors utilizing microspheres. Waft, et al US/20030016897 Jan. 23, 2003
31) Use of a cell-based, lawn format assay to rapidly screen a 442,368 bead-based peptide library. Jayawickreme, et al J Pharmacol Toxicol Methods. December 1999;42(4):189-97
32) Steric factors influencing hybridization of nucleic acids to oligonucleotide arrays. Shchepinov, et al Nucleic Acids Res. Mar. 15, 1997;25(6):1155-61
33) Method and apparatus for performing large numbers of reactions using array assembly with releasable primers. Brennan, et al U.S. Pat. No. 6,632,641 Oct. 14, 2003
34) Conjugates of antisense oligonucleotides with the Tat and antennapedia cell-penetrating peptides: effects on cellular uptake, binding to target sequences, and biologica actions. Astriab-Fisher, et al Pharm Res June 2002;19(6):744-754
35) Conjugate for efficient delivery of short interfering RNA (siRNA) into mammalian cells. Muratovska A, et al FEBS Left. Jan. 30, 2004;558(1-3):63-8
36) Advancing molecular therapies through in vivo bioluminescent imaging. (review) McCaffrey A, et al Mol Imaging. April 2003;2(2):75-86

1) A combinatorial library, each member a multiple-ligand-conjugated nucleic acid which is affixed on or within a support, and comprises a small bioactive nucleic acid.

2) The library as described in claim 2 wherein for each member ligand combination was effected via hybridization of oligonucleotides.

3) The library as described in claim 1, each member an oligonucleotide hairpin whose terminal nucleotides are conjugated to separate ligands.

4) The library as described in claim 3 wherein the support is an encoded particle.

5) The library as described in claim 3 wherein the members are arrayed.

6) The library as described in claim 3 wherein the members are affixed covalently.

7) A library as described in claim 1, the nucleic acid of each member consisting of multiple hybridized oligonucleotides.

8) The library as described in claim 7 wherein the support is an encoded particle.

9) The library as described in claim 7 wherein the members are arrayed.

10) The library as described in claim 7 wherein the members are affixed covalently.

11) A combinatorial library, each member a multiple-ligand-conjugated nucleic acid which is covalently affixed on or within a support.

12) The library as described in claim 11 wherein for each member ligand combination was effected via hybridization of oligonucleotides.

13) The library as described in claim 11 wherein the members are arrayed, and each member is an oligonucleotide hairpin whose terminal nucleotides are conjugated to separate ligands.

14) The library as described in claim 13 wherein the ligand set of each member is, or is anticipated to bind a target and thereby induce a cellular response.

15) The library as described in claim 13 wherein the ligand set of each member is, or is anticipated to bind a T cell receptor or antibody.

16) The library as described in claim 13 wherein the ligand set of each member binds, or is anticipated to bind to a site on a protein which interfaces with another protein.

17) The library as described in claim 11 wherein the members are arrayed, and the nucleic acid of each consists of multiple hybridized oligonucleotides.

18) The library as described in claim 17 wherein the ligand set of each member is, or is anticipated to bind a target and thereby induce a cellular response.

19) The library as described in claim 17 wherein the ligand set of each member is, or is anticipated to bind a T cell receptor or antibody.

20) The library as described in claim 12 wherein the ligand set of each member binds, or is anticipated to bind to a site on a protein which interfaces with another protein.
